(19) United States
(12) Patent Application Publication
(10) Pub. No.: US 2009/0198109 A1
(43) Pub. Date: Aug. 6, 2009

(54) Method and apparatus for guided operating instruction for physiological measuring instrument

(75) Inventor: Shan-Yi Yu, Taipei County (TW)

Correspondence Address: BACON & THOMAS, PLLC, 625 SLATERS LANE, FOURTH FLOOR, ALEXANDRIA, VA, 22314-1176, US

(73) Assignee: HEALTH & LIFE CO., LTD., CHUNG HO CITY (TW)

(21) Appl. No.: 12/068,386

(22) Filed: Feb. 6, 2008

(51) Int. Cl. A61B 5/00

(52) U.S. Cl. 600/300

(57) ABSTRACT

In a method and an apparatus of a guided operating instruction for a physiological measuring instrument, the apparatus includes: a display area, for displaying detected, computed and recorded physiological measurement information; an operating area, having function keys, and the operating area further includes: a symbol layer, having independent specific symbol blocks for displaying the function keys; a light source indicating layer, having specific light source blocks corresponding to the specific symbol blocks, and emitting a light source at the specific light source block; a key pressing circuit layer, having a touch control circuit block corresponding to the specific light source block of the light source indicating layer. The physiological measuring instrument only receives signals of the touch control circuit block at a specific position area that emits a light source with the light source indicating layer, so as to guide users to complete a physiological measurement quickly and easily.

### BACKGROUND OF THE INVENTION

1. Field of the Invention

The present invention relates to a method and an apparatus of a guided operating instruction for a physiological measuring instrument, and more particularly to a method and an apparatus that combine a symbol display with a function key, and use the symbol display to give instructions or guide the measuring operations at different stages, so as to guide users to quickly and intuitively carry out the operations in a simple and easy manner.

2. Description of the Related Art

As the life span of modern people is generally prolonged and its major factor resides on the well-developed modern medical technologies and instruments. The health care concept and the willingness of accepting medical treatments are improved, and thus “Prevention rather than treatment” and “Home health care” can be implemented into our daily life to achieve the positive maintenance of our health and the best effect of controlling our physical conditions. At present, various different physiological measuring instruments (such as a sphygmomanometer) provide a convenient way for people to measure their physiological conditions at home and know better about the physiological information, so that appropriate measures can be taken in advance to maintain our health and prevent illness from getting worse. Since existing physiological measuring instruments intend to make accurate measurements and display the interpretation of data, most of these instruments are electronic devices, and the casing and the panel of these physiological measuring instruments generally include an operating panel and a data display screen, and the operating panel comes with complicated operating keys provided for the selection and operation of various different measuring functions, and the data display screen is provided for displaying the results of the measured physiological data. However, the physiological measuring instruments of this sort are used mostly by patients and the elderly. If the physiological measuring instruments come with many functions and keys, the patients and elderly may be confused on the complicated selection and operation, and thus a clear instruction manual and a certain period of time are needed to get familiar with the use of these measuring instruments. Obviously, such instruments are not user-friendly, and their design does not allow users to make measurements quickly and easily, and thus the conventional physiological measuring instruments demand immediate attentions and feasible solutions.

In view of the foregoing shortcomings of the prior art physiological measuring instrument, the inventor of the present invention based on years of experience in the related industry to conduct extensive experiments and researches, and finally invented a method and an apparatus of a guided operating instruction for physiological measuring instrument to overcome the shortcomings of the prior art.

### SUMMARY OF THE INVENTION

It is a primary objective of the present invention to overcome the foregoing shortcomings of the prior art by providing a method and an apparatus of a guided operating instruction for a physiological measuring instrument that combine a symbol display with a superimposed function key area, so that the operation of a selected function shows symbol displays at different stages to facilitate users to press the keys according to the displayed symbols, so as to quickly and intuitively guide users to complete the measurements in a simple and easy manner.

To achieve the foregoing objective and effect, the technical measures taken by the present invention includes: a casing, in the shape of a case; a microprocessing unit, installed in the casing, for computing and recording physiological information; a display area, disposed on a surface of the casing, and electrically coupled to the microprocessing unit, for displaying information provided by the microprocessing unit; an operating area, disposed on a surface of the casing, and electrically coupled to the microprocessing unit, and having a plurality of function keys, and the operating area that are superimposed further comprises: a symbol layer, having a plurality of specific symbol blocks independent with each other for displaying the function keys, and the specific symbol block having a specific symbol; a light source indicating layer, including a plurality of specific light source blocks corresponding to the specific symbol blocks, for receiving a signal of the microprocessing unit and emitting a light source at the specific light source block to display the specific symbol; a key pressing circuit layer, having a touch control circuit block corresponding to the specific light source block of the light source indicating layer, and the microprocessing unit only receiving a signal from the touch control circuit block at the specific position area where the light source indicating layer emitting a light source at the same time. The method of the present invention comprises the following steps: (1) Start the physiological measuring instrument to set to a standby mode, and display at least one first stage function key; (2) Perform selecting a key operation according to the displayed first stage function key; (3) Produce a first stage functional operation or display at least one second stage function key at the same time; (4) Perform selecting an operation according to the displayed function key at each stage until all measurements are finished.

The first stage function key of Step (1) and the second stage function key of Step (3) show function symbols at the superimposed position.

After Step (4) is finished, a measuring ON/OFF key is pressed to set to an off mode or to turn off the physiological measuring instrument automatically after a predetermined time.

To make it easier for the examiner to understand the object, shape, structure, instrument, characteristics and functions of this invention, the specification accompanied by the drawings is described as follows.

### BRIEF DESCRIPTION OF THE DRAWINGS

FIG. 1 is a schematic view of the present invention;

FIG. 2 is a schematic view of a surface of a casing of the present invention;

FIG. 3 is a schematic view of a structure of an operating area of the present invention;

FIG. 4 is a schematic view of another structure of an operating area of the present invention;

FIG. 5 is a schematic view of a sphygmomanometer that is set to an OFF mode in accordance with a preferred embodiment of the present invention;

FIG. 6 is a schematic view of a sphygmomanometer that is set to a standby mode in accordance with a preferred embodiment of the present invention;

FIG. 7 is a schematic view of a sphygmomanometer that is set to an ON mode in accordance with a preferred embodiment of the present invention; and

FIG. 8 is a flow chart of an operating method of the present invention.

### DETAILED DESCRIPTION OF THE PREFERRED EMBODIMENTS

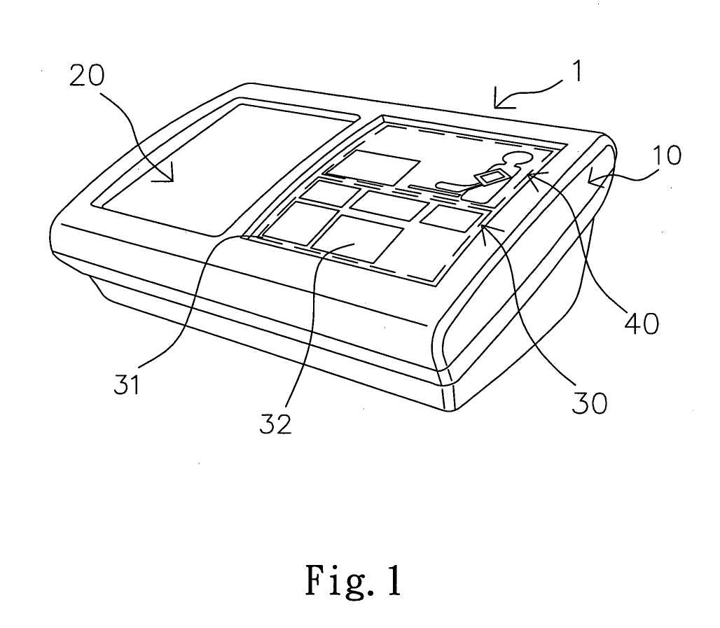

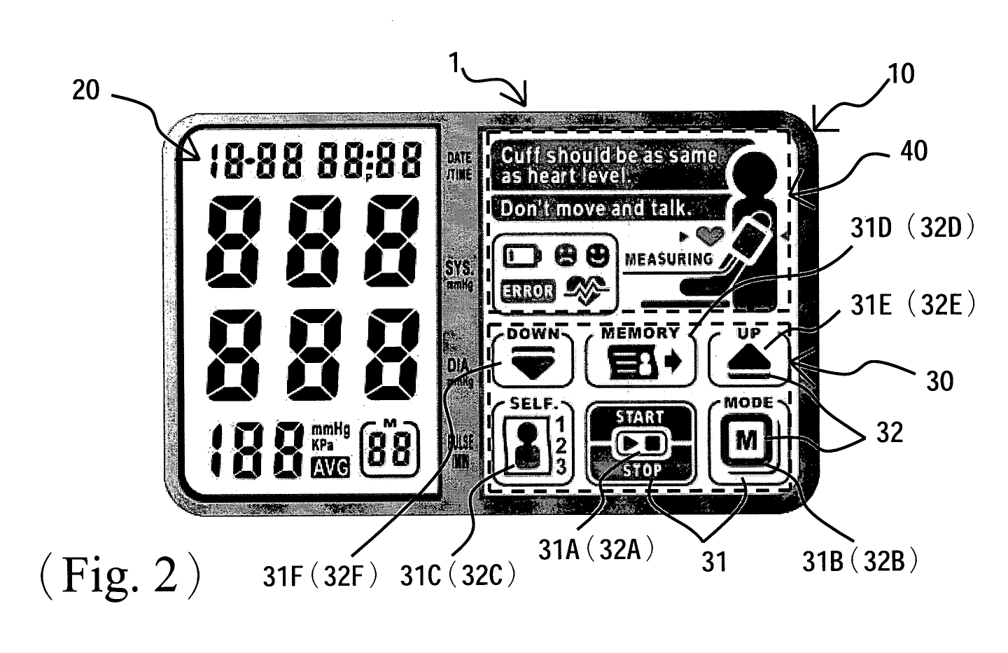

Referring to FIGS. 1 and 2 for a guided operating instruction apparatus of a physiological measuring instrument for measuring human physiological information, a sphygmomanometer 1 is used as the physiological measuring instrument for illustrating the invention. The sphygmomanometer 1 comprises a casing 10, a display area 20, an operating area 30 and a help area 40. The casing 10 is in the shape of a case, and the casing 10 includes a microprocessing unit (which is a microprocessor MCU in this embodiment) and a circuit control device (not shown in the figure). The microprocessing unit is provided for computing and recording physiological information. The display area 20 (or display screen) is disposed on a surface of the casing 10 and electrically coupled to the microprocessing unit for displaying the information such as the data provided by the microprocessing unit.

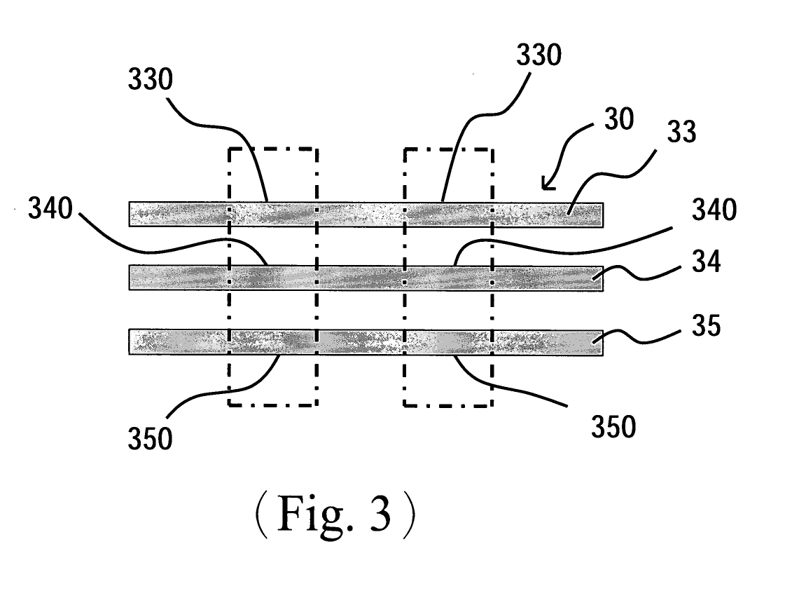

The operating area 30 is disposed on a surface of the casing 10 and electrically coupled to the microprocessing unit, and the operating area 30 includes a plurality of function keys 31 for displaying function symbols 32. Referring to FIG. 3 for the illustration of the physical structure of the operating area 30, and the operating area 30 further comprises a symbol layer 33, a light source indicating layer 34 and a key pressing circuit layer 35, that are superimposed with each other. The symbol layer 33 includes a plurality of specific symbol blocks 330 that are independent with each other, and the specific symbol block 330 includes a specific symbol such as a text or a pattern formed on a specific position of the symbol layer 33 in advance by printing, engraving or other methods. The light source indicating layer 34 is superimposed under the symbol layer 33, for receiving a signal from the microprocessing unit and emits a light source at the specific light source block 340 of the light source indicating layer 34 to display a specific symbol. The specific light source block 340 corresponds to the specific symbol block 330 of the symbol layer 33, such that the function symbol 32 (formed by the specific symbol block 330) can be displayed on the operating area 30 by the projection of the light source. In other words, the function key 31 can be displayed. The key pressing circuit layer 35 is superimposed under the light source indicating layer 34, and a touch control circuit block 350 is disposed on the key pressing circuit layer 35 and corresponding to the specific light source block 340 of the light source indicating layer 34, for generating touch control sensing circuit signals, and the change of the circuit signals is sent to the microprocessing unit for the determination and processing, and the microprocessing unit only receives signals from a touch control circuit block 350 at a specific position area 340 that emits a light source together with the indicating layer 34.

Since the symbol layer 33, the light source indicating layer 34 and the key pressing circuit layer 35 of the operating area 30 are superimposed with each other, therefore when the specific symbol block 330 of the symbol layer 3 is pressed, the touch control circuit block 350 of the key pressing circuit layer 35 is also pressed for generating touch control sensing circuit signals and transmitting the circuit signals. And then, a feedback signal of the microprocessing unit is provided to continue producing a different function symbol 32 for the next stage, and the light source of the function key 31 is projected for the display for the next stage. In other words, the specific symbol of the function symbol 32 is shown on the surface of the operating area 30 (or the function key 31) by the projection of the light source.

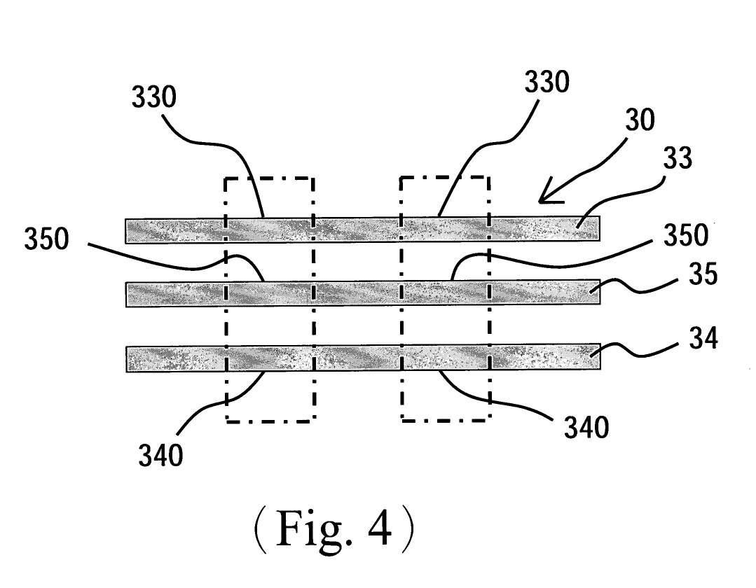

Further, the operating area 30 comprises the symbol layer 33, the light source indicating layer 34 and the key pressing circuit layer 35 that are superimposed with each other, and such superimposed position can be adjusted as shown in FIG. 4, wherein the key pressing circuit layer 35 is disposed under the symbol layer 33, and the light source indicating layer 34 is disposed under the key pressing circuit layer 35.

The function key 31 of the operating area 30 further comprises a measuring ON/OFF key 31A, a mode key 31B, a user switching key 31C, a memory function key 31D, an UP key 31E and a DOWN key 31F. The measuring ON/OFF key 31A, the mode key 31B, the user switching key 31C, the memory function key 31D, the UP key 31E and the DOWN key 31F display correspond to the function symbols 32A-32F respectively.

The help area 40 is disposed on a surface of the casing (wherein the help area 40 of this embodiment is separated from the display area, but the help area and the display area can be combined if needed) and electrically coupled to the microprocessing unit. The help area 40 includes a text or a symbol for showing different information, and such information includes the blood pressure measurement guide, error messages, low power conditions, and all kinds of information interactive with the users.

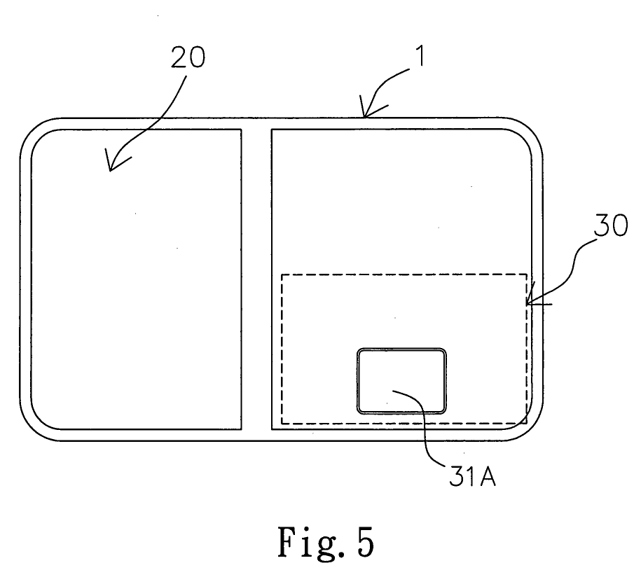

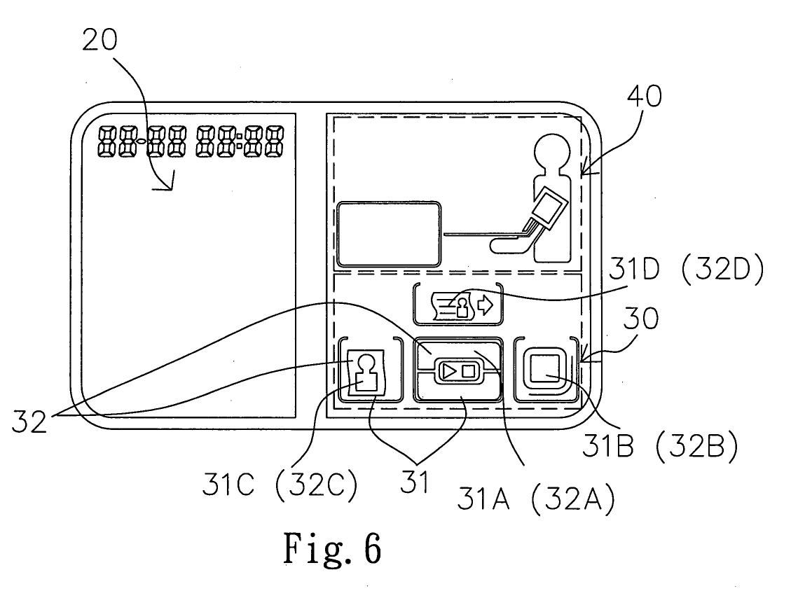

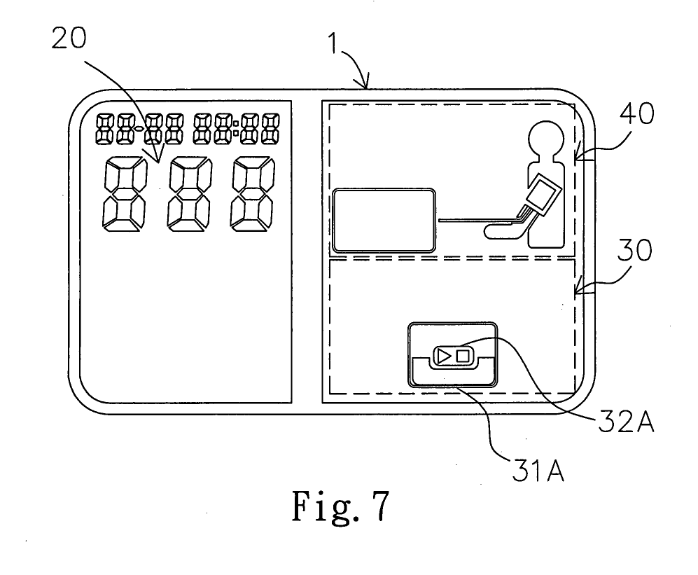

Referring to FIGS. 5 to 7 for the application and operation of the sphygmomanometer 1 in accordance with a preferred embodiment, FIG. 5 shows that when the sphygmomanometer 1 is at an off mode, the position (or the frame) of the measuring ON/OFF key 31A is shown. If the measuring ON/OFF key 31A is pressed to turn on the sphygmomanometer 1 (at a standby mode) and display the measuring ON/OFF key 31A, the mode key 31B, the user switching key 31C and the memory function key 31D (including the function symbols 32A-32D thereon) of the operating area 30 simultaneously as shown in FIG. 6 for the selection of the functional operations for the next stage. If a START position of the measuring ON/OFF key 31A is selected and pressed, the blood pressure measurement will start, and the measuring ON/OFF key 31A (including the function symbol 32A thereon) will show up as shown in FIG. 7. When the measurement of blood pressures starts, the help area 40 will display corresponding messages in texts or symbols to remind users about the current status and cautions.

If the START position of the measuring ON/OFF key 31A is pressed, one function key 31 the measuring ON/OFF key 31A is displayed for users to select the operation of the next stage. If a STOP position of the measuring ON/OFF key 31A is pressed now, the measurement of blood pressures will stop and the sphygmomanometer 1 will return to its standby mode as shown in FIG. 6. After blood pressure measurements are taken, the measured results (readings) will be displayed in the display area 20, and users may select pressing the STOP position of the measuring ON/OFF key 31A to return the sphygmomanometer 1 to its standby mode (as shown in FIG. 6).

Of course, the measuring ON/OFF key 31A, the mode key 31B, the user switching key 31C and the memory function key 31D of the operating area 30 at the standby mode as shown in FIG. 6 and corresponding to the projection of the light source of the light source indicating layer 34 can be displayed, so that the function symbols 32A-32D can be displayed to facilitate users to select the operations as illustrated in FIGS. 5 to 7. Other than the measuring ON/OFF key 31A, the same operating principle applies to the displays of the mode key 31B, the user switching key 31C and the memory function key 31D at the standby mode. After a selected key is pressed, the function keys 31 and the function symbols 32 for the coming stages can be displayed, so that users can follow the symbol displayed position of the light source to complete the required operations. The present invention guides users to complete different functional applications in a simple and easy manner.

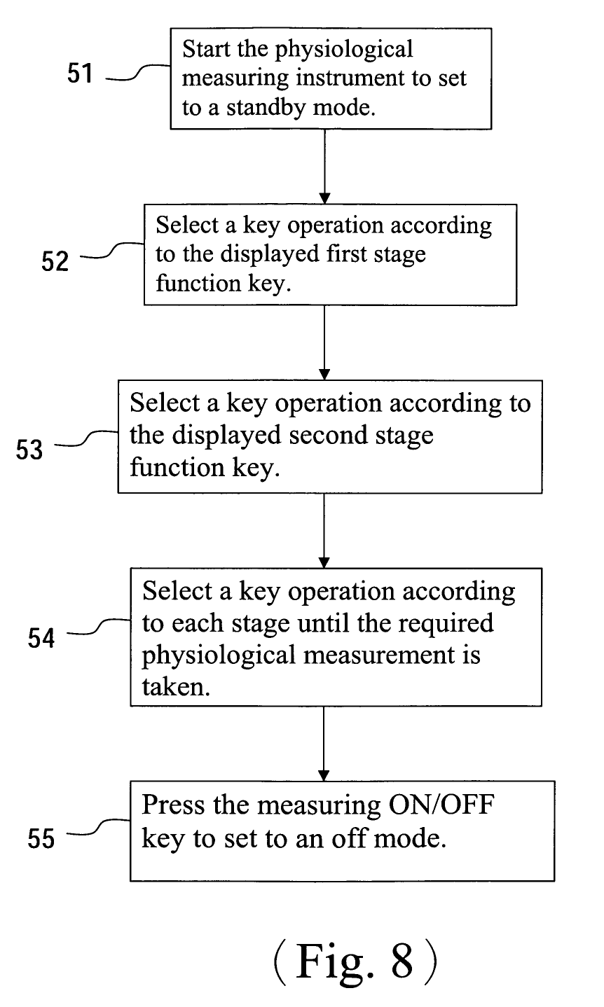

Referring to FIG. 8 for the flow chart illustrating a guided operating instruction method for a physiological measuring instrument in accordance with the present invention, the method comprises the following steps:

(1) Start the physiological measuring instrument to set to a standby mode 51 by pressing the measuring ON/OFF key 31A to show at least one first stage function key 31 and a function symbol 32, and the function symbol 32 is superimposed on the function key 31 at a same position.

(2) Perform selecting a press operation according to the displayed function key 52. In other words, the function key 31 of a desired function is pressed to produce a first stage functional operation or simultaneously display at least one second stage function key 31 and function symbol 32. Of course, the function symbol 32 is superimposed with the function key 31 at the same position.

(3) Select the key operation according to the displayed second stage function key 53. In other words, a selected second stage function key 31 is pressed to produce a functional operation or simultaneously display at least one third stage function key 31 and function symbol 32;

(4) Select the operation according to the function key displayed at each stage until the required physiological measurement is taken 54; and

(5) The function key has a measuring ON/OFF key and press the measuring ON/OFF key to turn off the measuring instrument 55 or to turn off the physiological measuring instrument automatically after a predetermined time.

In the present invention, the operating area includes the symbol layer, the light source indicating layer and the key pressing circuit layer superimposed with each other, so that the light of the light source of the light source indicating layer can be projected to show the specific symbols on the symbol layer, and the position of the specific symbol is exactly the position of the function key. The symbol display can be used for selecting a function key at the same position, and the function symbols for the coming stages are displayed to guide users to quickly and intuitively carry out the operation in a simple and easy manner, so as to achieve the convenience of selecting the operation.

In summation of the above description, the present invention enhances the performance than the conventional structure and further complies with the patent application requirements and is duly filed for patent application. While the invention has been described by means of specific embodiments, numerous modifications and variations could be made thereto by those skilled in the art without departing from the scope and spirit of the invention set forth in the claims.

What is claimed is:

1. A guided operating instruction apparatus for a physiological measuring instrument, applied for measuring human physiological information, comprising:
a casing, being in the shape of a case, and having a microprocessing unit for computing and recording said physiological information;
a display area, disposed on a surface of said casing, and electrically coupled to said microprocessing unit, for displaying information provided by said microprocessing unit;
an operating area, disposed on the surface of said casing, and electrically coupled to said microprocessing unit, and having a plurality of function keys, and said operating area further superimposedly comprising:
a symbol layer, having a plurality of specific symbol blocks independent with each other for displaying said function keys, and each specific symbol block having a specific symbol;
a light source indicating layer, having a plurality of specific light source blocks corresponding to said specific symbol blocks, and said light source indicating layer being provided for receiving signals of said microprocessing unit and emitting a light source from at least one of said specific light source blocks; and
a key pressing circuit layer, having a touch control circuit block corresponding to said specific light source block of said light source indicating layer, and said microprocessing unit receiving a signal from said touch control circuit block at said specific position area of said light source indicating layer to emit a light source.

2. The guided operating instruction apparatus for a physiological measuring instrument according to claim 1, wherein said key pressing circuit layer falls between said symbol layer and said light source indicating layer.

3. The guided operating instruction apparatus for a physiological measuring instrument according to claim 1, wherein said light source indicating layer falls between said symbol layer and said key pressing circuit layer.

4. The guided operating instruction apparatus for a physiological measuring instrument according to claim 1, wherein said specific symbol of said symbol layer is a text or a pattern.

5. The guided operating instruction apparatus for a physiological measuring instrument according to claim 1, wherein said specific symbol of said symbol layer is displayed on the same position of said function key by projecting a light source of said specific light source block.

6. The guided operating instruction apparatus for a physiological measuring instrument according to claim 1, wherein said microprocessing unit comprises a microprocessor (MCU) and a circuit control device.

7. The guided operating instruction apparatus for a physiological measuring instrument according to claim 1, wherein said specific symbol is formed on a specific position of said symbol layer in advance by printing, engraving or other methods.

8. The guided operating instruction apparatus for a physiological measuring instrument according to claim 1, further comprising a help area disposed on a surface of said casing and electrically coupled to said microprocessing unit.

9. The guided operating instruction apparatus for a physiological measuring instrument according to claim 8, wherein said help area is also a display area.

10. The guided operating instruction apparatus for a physiological measuring instrument according to claim 8, wherein said help area comprises a text or a symbol for showing different information.

11. A guided operating instruction method for a physiological measuring instrument, applied to the apparatus of claim 1, comprising the steps of:
(1) starting said physiological measuring instrument to set to a standby mode, and displaying at least one first stage function key;
(2) selecting a key operation according to said displayed first stage function key;
(3) producing a functional operation of said first stage or simultaneously displaying at least one second stage function key; and
(4) selecting an operation according to said function key displayed at each stage until the final measurement is taken.

12. The guided operating instruction method for a physiological measuring instrument according to claim 11, wherein said first stage function key of Step (1) and said second stage function key of Step (3) are superimposed at the same position to show different function symbols.

13. The guided operating instruction method for a physiological measuring instrument according to claim 11, wherein said function key includes a measuring ON/OFF key and said measuring ON/OFF key is pressed after Step (4) to set said physiological measurement instrument to an OFF mode.

14. The guided operating instruction method for a physiological measuring instrument according to claim 11, wherein said physiological measuring instrument is turned off automatically within a predetermined time after Step (4) is completed.
